(19) United States
(12) Patent Application Publication
(10) Pub. No.: US 2023/0124687 A1
(43) Pub. Date: Apr. 20, 2023

(54) METHOD FOR CALIBRATING ENDOSCOPE AND ENDOSCOPE SYSTEM

(71) Applicant: OLYMPUS MEDICAL SYSTEMS CORP., Tokyo (JP)

(72) Inventors: Katsuhiko YOSHIMURA, Tokyo (JP); Kosuke KISHI, Mitaka-shi (JP)

(73) Assignee: OLYMPUS MEDICAL SYSTEMS CORP., Tokyo (JP)

(21) Appl. No.: 17/501,194

(22) Filed: Oct. 14, 2021

(51) Int. Cl. A61B 1/00; G02B 23/24; A61B 1/05

(52) U.S. Cl. CPC A61B 1/00057; G02B 23/2484; A61B 1/05

(57) ABSTRACT

The method for calibrating an endoscope is a method for calibrating an endoscope having a curved portion capable of being driven by bending, and includes: an arrangement step of suspending and arranging a distal end portion of the endoscope including the curved portion; a bending step of bending the curved portion of the distal end portion which has been suspended; a measurement step of measuring shape information of the curved portion; and an adjustment step of updating a control parameter by which the endoscope drives the curved portion, based on the shape information which has been measured.

### TECHNICAL FILED

The present invention relates to a method for calibrating an endoscope and an endoscope system.

### BACKGROUND

Conventionally, an endoscope system including an endoscope has been used for observation in a luminal organ such as a digestive tract and treatment in open surgery. The endoscope includes a curved portion capable of being driven by bending. Although the endoscope is supplied in a sufficiently calibrated state, it may be necessary to adjust the operation of the curved portion due to aging or the like. In that case, the user needs to calibrate the endoscope using a calibration jig, as described in PCT International Publication No. WO 2016/136353 and the like.

### SUMMARY

The method for calibrating an endoscope according to a first aspect of the present disclosure is a method for calibrating an endoscope having a curved portion capable of being driven by bending, and includes: an arrangement step of suspending and arranging a distal end portion of the endoscope including the curved portion; a bending step of bending the curved portion of the distal end portion which has been suspended; a measurement step of measuring shape information of the curved portion; and an adjustment step of updating a control parameter by which the endoscope drives the curved portion, based on the shape information which has been measured.

The endoscope system according to a second aspect of the present disclosure includes an endoscope having a curved portion capable of being driven by bending, and a control device configured to control driving of the curved portion, wherein the control device performs a bending step of bending the curved portion, a measurement step of measuring shape information of the curved portion, and an adjustment step of updating a control parameter by which the endoscope drives the curved portion, based on the shape information which has been measured.

### BRIEF DESCRIPTION OF THE DRAWINGS

FIG. 1 is an overall view of an electric endoscope system according to a first embodiment.

FIG. 2 is a diagram showing an endoscope and an operation device of the electric endoscope system used by the operator.

FIG. 3 is a diagram showing an insertion portion of the endoscope.

FIG. 4 is a cross-sectional view of a part of a curved portion of the endoscope.

FIG. 5 is an enlarged view of a knot ring of the curved portion in region E shown in FIG. 4.

FIG. 6 is a cross-sectional view of the curved portion along the C1-C1 line of FIG. 4 and FIG. 5.

FIG. 7 is a functional block diagram of a driving device of the electric endoscope system.

FIG. 8 is a functional block diagram of a video control device of the electric endoscope system.

FIG. 9 is a functional block diagram of a main controller of the electric endoscope system.

FIG. 10 is a control flowchart of a main controller of a control device in the electric endoscope system.

FIG. 11 is a diagram showing the endoscope with its distal end suspended.

FIG. 12 is a control flowchart of the main controller of the control device in the electric endoscope system according to a second embodiment.

FIG. 13 is a diagram showing a sheet-shaped marker as an example of a marker.

FIG. 14 is a diagram showing a box-shaped marker as an example of a marker.

### DETAILED DESCRIPTION

### First Embodiment

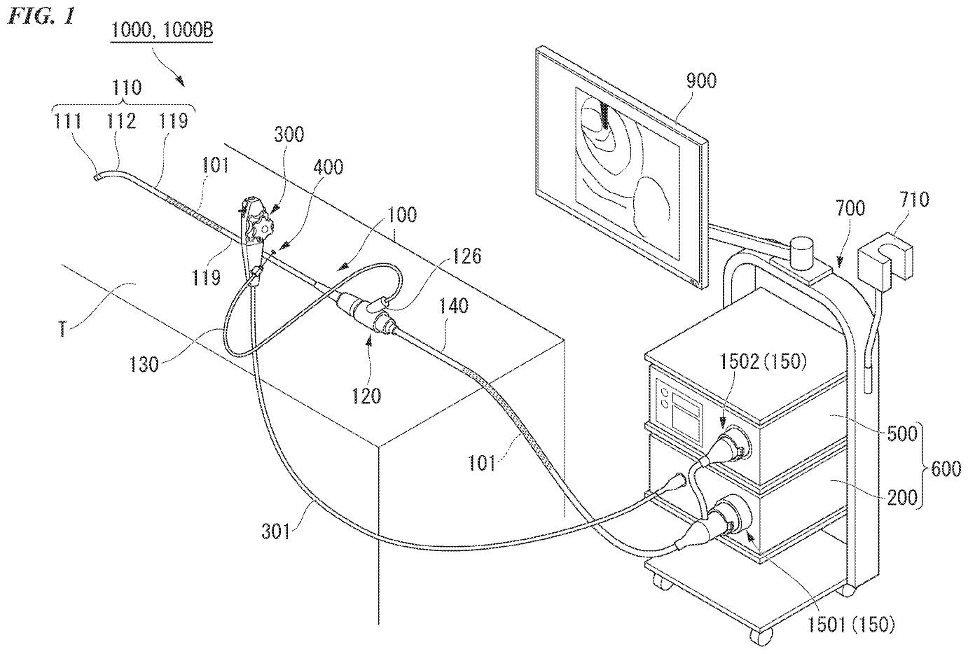

An electric endoscope system 1000 according to a first embodiment of the present disclosure will be described with reference to FIG. 1 to FIG. 17. FIG. 1 is an overall view of the electric endoscope system 1000 according to the present embodiment. The electric endoscope system 1000 is an example of a manipulator system.

### [Electric Endoscope System 1000]

The electric endoscope system 1000 is a medical system used to observe and treat the inside of a patient P as shown in FIG.1. Further, the electric endoscope system 1000 is a medical system that maintains an endoscope 100. The electric endoscope system 1000 includes the endoscope 100, a driving device 200, an operation device 300, a treatment tool 400, a video control device 500, a display device 900, and an accommodation rack 700.

The endoscope 100 is a device that is inserted into the lumen of the patient P to observe and treat the affected area. The endoscope 100 is detachable from the driving device 200. An internal path 101 is formed inside the endoscope 100. In the following description, in the endoscope 100, the side inserted into the lumen of the patient P is referred to as the “distal end side (A1)”, and the side mounted on the driving device 200 is referred to as the “proximal end side (A2)”.

The driving device 200 is detachably connected to the endoscope 100 and the operation device 300. The driving device 200 drives the built-in motor to electrically drive the endoscope 100 based on the operation input to the operation device 300. Further, the driving device 200 drives a built-in pump or the like based on the operation input to the operation device 300 to cause the endoscope 100 to perform air supply suction.

The operation device 300 is detachably connected to the driving device 200 via an operation cable 301. The operation device 300 may be capable of communicating with the driving device 200 by wireless communication instead of wired communication. The operator S can electrically drive the endoscope 100 by operating the operation device 300.

The treatment tool 400 is a device for treating the affected portion by inserting the internal path 101 of the endoscope 100 into the lumen of the patient P. In FIG. 1, the treatment tool 400 is inserted into the internal path 101 of the endoscope 100 via an extension channel tube 130. The treatment tool 400 may be inserted directly from a forceps opening 126 into the internal path 101 of the endoscope 100 without passing through the extension channel tube 130.

The video control device 500 is detachably connected to the endoscope 100, and acquires a captured image from the endoscope 100. The video control device 500 causes the display device 900 to display the captured image acquired from the endoscope 100 and a GUI image or CG image for the purpose of providing information to the operator.

The driving device 200 and the video control device 500 constitute a control device 600 that controls the electric endoscope system 1000. The control device 600 may further include a peripheral device such as a video printer. The driving device 200 and the video control device 500 may be an integrated device.

The display device 900 is a device capable of displaying an image such as an LCD. The display device 900 is connected to the video control device 500 via a display cable 901.

The driving device 200, the video control device 500, and the display device 900 are accommodated in the accommodation rack 700. The accommodation rack 700 is equipped with tires and is easy to move. The accommodation rack 700 is provided with a hanger (trolley) 710 that can be installed by suspending the endoscope 100.

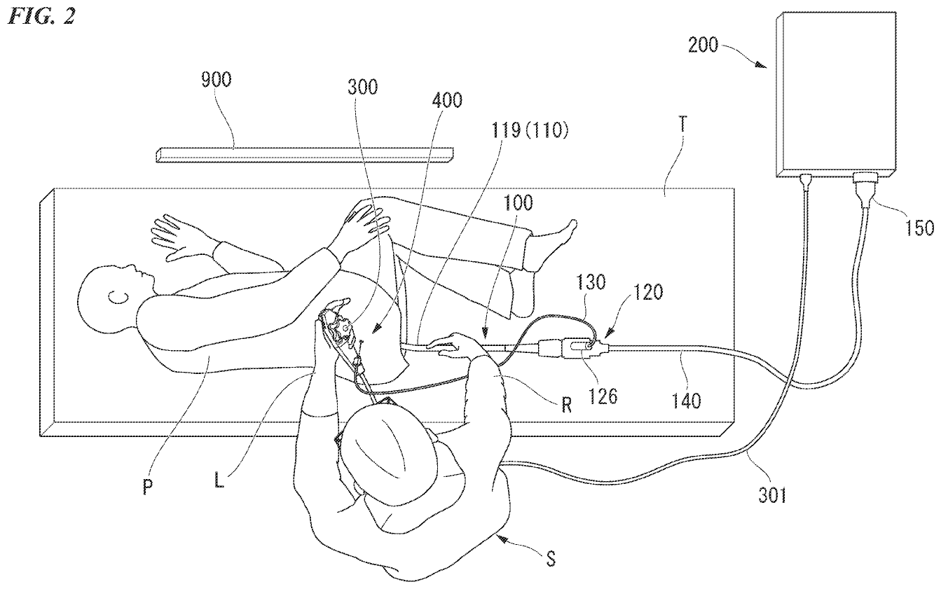

FIG. 2 is a diagram showing the endoscope 100 and the operation device 300 used by the operator S.

For example, the operator S operates the endoscope 100 inserted into the lumen from the anus of the patient P with the right hand R while observing the captured image displayed on the display device 900, and operates the operation device 300 with the left hand L. Since the endoscope 100 and the operation device 300 are separated, the operator S can operate the endoscope 100 and the operation device 300 independently without either affecting the other.

### [Endoscope 100]

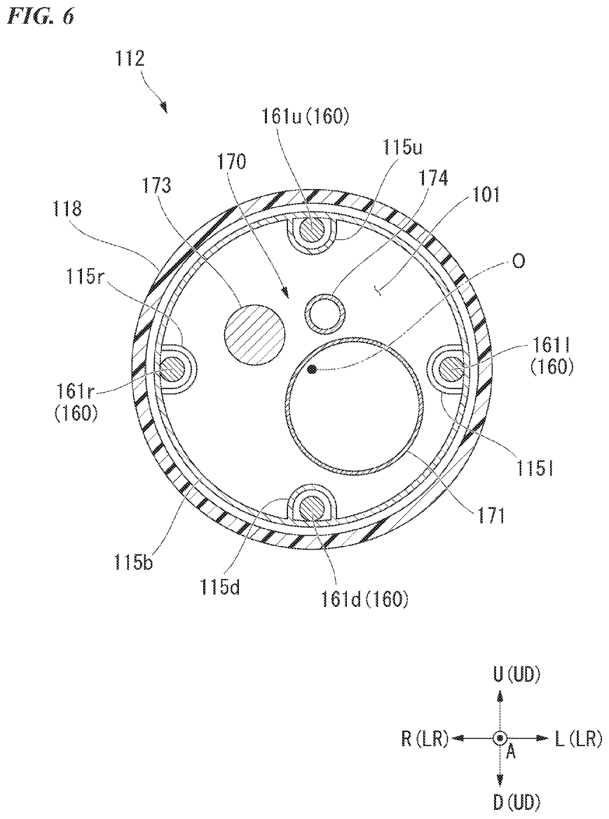

As shown in FIG. 1, the endoscope 100 includes an insertion portion 110, a connecting portion 120, an extracorporeal soft portion 140, an attachment/detachment portion 150, a curved wire 160 (see FIG. 6), and an internal object 170 (see FIG. 6). The insertion portion 110, the connecting portion 120, the extracorporeal soft portion 140, and the attachment/detachment portion 150 are connected in order from the distal end side. The connecting portion 120 can connect the extension channel tube 130.

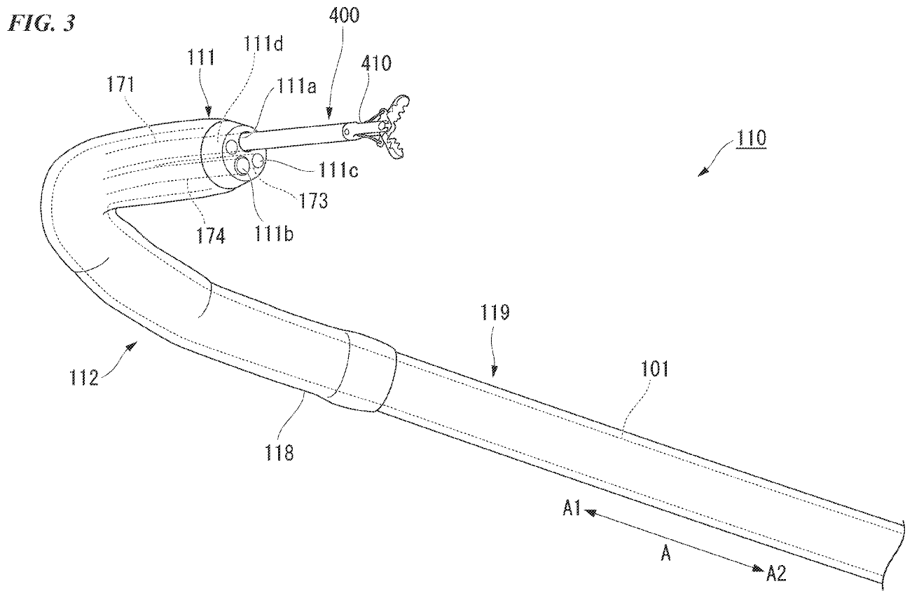

FIG. 3 is a diagram showing the insertion portion 110 of the endoscope 100.

Inside the endoscope 100, the internal path 101 is formed that extends from the distal end of the insertion portion 110 to the proximal end of the attachment/detachment portion 150 along the longitudinal direction A of the endoscope 100. The curved wire 160 and the internal object 170 are inserted into the internal path 101.

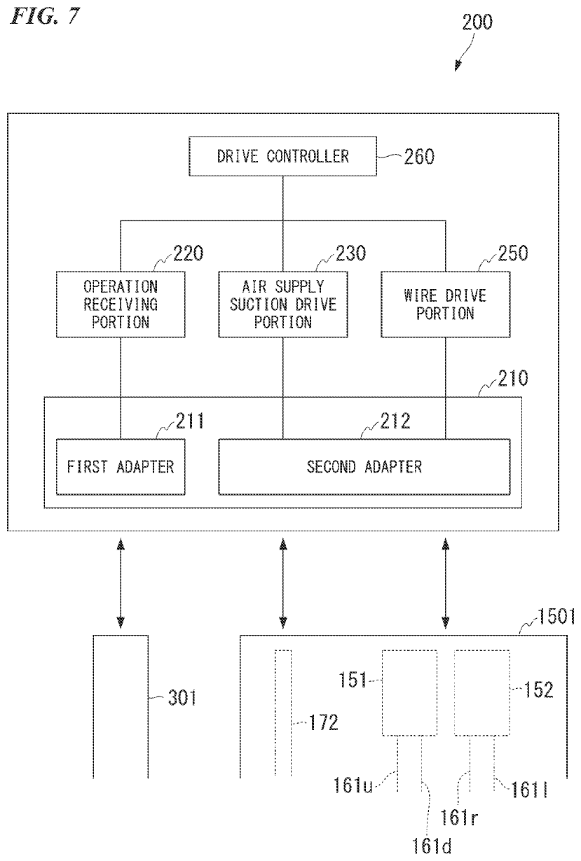

The internal object 170 includes a channel tube 171, an air supply suction tube 172 (see FIG. 7), an imaging cable 173, and a light guide 174.

### [Insertion Portion 110]

The insertion portion 110 is an elongated long member that can be inserted into the lumen. The insertion portion 110 has a distal end portion 111, a curved portion 112, and an internal soft portion 119. The distal end portion 111, the curved portion 112, and the internal soft portion 119 are connected in order from the distal end side.

The distal end portion 111 is formed of a metal or the like into a substantially cylindrical shape. As shown in FIG. 3, the distal end portion 111 has an opening portion 111a, an illumination portion 111b, and an imaging portion 111c. The opening portion 111a is an opening that communicates with the channel tube 171. As shown in FIG. 3, a treatment portion 410 such as a gripping forceps provided at the distal end of the treatment tool 400 through which the channel tube 171 is inserted protrudes from the opening portion 111a.

The illumination portion 111b is connected to the light guide 174 that guides the illumination light, and emits illumination light that illuminates an imaging target. The imaging portion 111c includes an imaging element such as CMOS, and images the imaging target. An imaging signal is transmitted to the video control device 500 via the imaging cable 173.

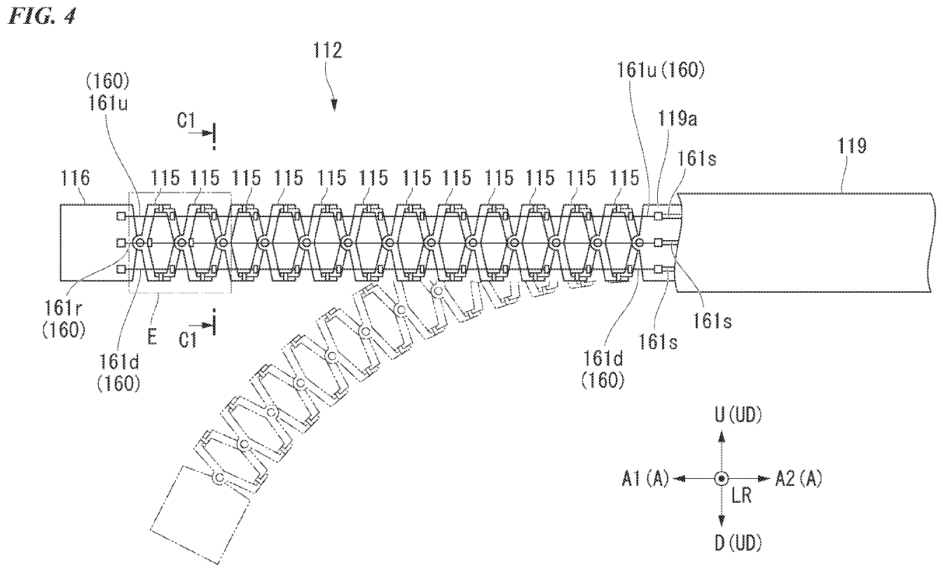

FIG. 4 is a view showing a part of the curved portion 112 as a cross-sectional view.

The curved portion 112 includes a plurality of knot rings (also referred to as curved pieces) 115, a distal end portion 116 connected to the distal ends of the plurality of knot rings 115, and an outer sheath 118 (see FIG. 3). The distal end portion 116, the plurality of knot rings 115, and a distal end portion 119a of the internal soft portion 119 are connected in the longitudinal direction A inside the outer sheath 118. The shape and number of the knot rings 115 included in the curved portion 112 are not limited to the shape and number of the knot rings 115 shown in FIG. 4.

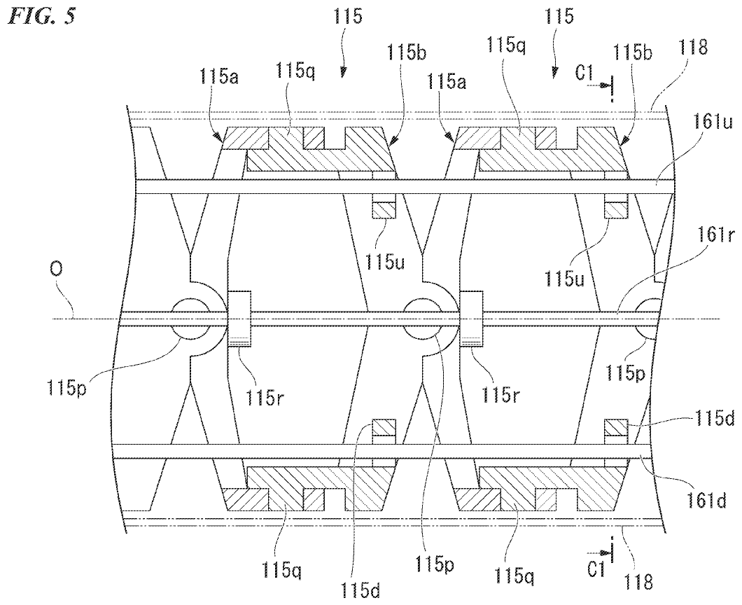

FIG. 5 is an enlarged view of the knot ring 115 in the region E shown in FIG. 4.

The knot ring 115 is a short cylindrical member made of metal. The plurality of knot rings 115 are connected so that the internal spaces of the adjacent knot rings 115 are continuous spaces.

Adjacent knot rings 115 are rotatably connected by a first rotation pin 115p in a vertical direction (also referred to as “UD direction”) perpendicular to the longitudinal direction A.

The knot ring 115 has a first knot ring 115a on the distal end side and a second knot ring 115b on the proximal end side. The first knot ring 115a and the second knot ring 115b are rotatably connected by a second rotation pin 115q in the left-right direction (also referred to as “LR direction”) perpendicular to the longitudinal direction A and the UD direction.

A first rotation pin 115p of the knot ring (curved piece) 115 is rotatable about a rotation axis extending in the LR direction. The second rotation pin 115q of the knot ring (curved piece) 115 is rotatable about a rotation axis extending in the UD direction.

The first knot ring 115a and the second knot ring 115b are alternately connected by the first rotation pin 115p and the second rotation pin 115q, and the curved portion 112 is bendable in a desired direction.

FIG. 6 is a cross-sectional view of the curved portion 112 along the C1-C1 line of FIG. 4 and FIG. 5.

An upper wire guide 115u and a lower wire guide 115d are formed on the inner peripheral surface of the second knot ring 115b. The upper wire guide 115u and the lower wire guide 115d are arranged on both sides in the UD direction with the central axis O in the longitudinal direction A interposed therebetween. A left wire guide 115l and a right wire guide 115r are formed on the inner peripheral surface of the first knot ring 115a. The left wire guide 115l and the right wire guide 115r are arranged on both sides in the LR direction with the central axis 0 in the longitudinal direction A interposed therebetween.

Through-holes through which the curved wire 160 is inserted are formed in the upper wire guide 115u, the lower wire guide 115d, the left wire guide 115l, and the right wire guide 115r along the longitudinal direction A.

The curved wire 160 is a wire that bends the curved portion 112. The curved wire 160 extends to the attachment/detachment portion 150 through the internal path 101. As shown in FIG. 4 and FIG. 6, the curved wire 160 has an upper curved wire 161u, a lower curved wire 161d, a left curved wire 161l, a right curved wire 161r, and four wire sheaths 161s.

As shown in FIG. 4, separate wire sheaths 161s are inserted into the upper curved wire 161u, the lower curved wire 161d, the left curved wire 161l, and the right curved wire 161r. The distal end of the wire sheath 161s is attached to the knot ring 115 at the proximal end of the curved portion 112. The wire sheath 161s extends to the attachment/detachment portion 150.

The upper curved wire 161u and the lower curved wire 161d are wires that bend the curved portion 112 in the UD direction. The upper curved wire 161u is inserted with the upper wire guide 115u, The lower curved wire 161d is inserted with the lower wire guide 115d,

As shown in FIG. 4, the distal ends of the upper curved wire 161u and the lower curved wire 161d are fixed to the distal end 116 of the distal end of the curved portion 112. The distal ends of the upper curved wire 161u and the lower curved wire 161d fixed to the distal end portion 116 are arranged on both sides in the UD direction with the central axis 0 in the longitudinal direction A interposed therebetween.

The left curved wire 161l and the right curved wire 161r are wires that bend the curved portion 112 in the LR direction. The left curved wire 161l is inserted with the left wire guide 115l. The right curved wire 161r is inserted with the right wire guide 115r.

The distal ends of the left curved wire 161l and the right curved wire 161r are fixed to the distal end 116 of the curved portion 112 as shown in FIG. 4. The distal ends of the left-curved wire 161l and the right-curved wire 161r fixed to the distal end portion 116 are arranged on both sides in the LR direction with the central axis O in the longitudinal direction A interposed therebetween.

The curved portion 112 can be bent in a desired direction by pulling or relaxing the curved wire 160 (upper curved wire 161u, lower curved wire 161d, left curved wire 161l, right curved wire 161r).

As shown in FIG. 6, the curved wire 160, the channel tube 171, the imaging cable 173, and the light guide 174 are inserted into the internal path 101 formed inside the curved portion 112.

The internal soft portion 119 is a long and flexible tubular member. The curved wire 160, the channel tube 171, the imaging cable 173, and the light guide 174 are inserted into an internal path 101 formed in the internal soft portion 119. At the distal end of the internal soft portion 119, a distal end portion 119a formed in a substantially cylindrical shape by a metal or the like is provided.

### [Connecting Portion 120]

As shown in FIG. 1, the connecting portion 120 is a member that connects the internal soft portion 119 and the extracorporeal soft portion 140 of the insertion portion 110. The connecting portion 120 includes the forceps opening 126, which is an insertion port for inserting the treatment tool 400 into the internal path 101.

### [Extracorporeal Soft Portion 140]

The extracorporeal soft portion 140 is a long tubular member. The curved wire 160, the imaging cable 173, the light guide 174, and the air supply suction tube 172 (see FIG. 7) are inserted into the internal path 101 formed inside the extracorporeal soft portion 140.

### [Attachment/detachment Portion 150]

As shown in FIG. 1, the attachment/detachment portion 150 includes a first attachment/detachment portion 1501 attached to the driving device 200 and a second attachment/detachment portion 1502 attached to the video control device 500. The first attachment/detachment portion 1501 and the second attachment/detachment portion 1502 may be an integral attachment/detachment portion.

The internal path 101 formed inside the extracorporeal soft portion 140 branches into the first attachment/detachment portion 1501 and the second attachment/detachment portion 1502. The curved wire 160 and the air supply suction tube 172 insert the first attachment/detachment portion 1501. The imaging cable 173 and the light guide 174 insert the second attachment/detachment portion 1502.

The first attachment/detachment portion 1501 has a tension sensor (not shown) that detects the tension of the curved wire 160 (upper curved wire 161u, lower curved wire 161d, left curved wire 161l, right curved wire 161r). The detection result of the tension sensor is acquired by a drive controller 260 of the driving device 200.

### [Driving Device 200]

FIG. 7 is a functional block diagram of the driving device 200.

The driving device 200 includes an adapter 210, an operation-receiving portion 220, an air supply suction drive portion 230, a wire drive portion 250, and the drive controller 260.

The adapter 210 has a first adapter 211 and a second adapter 212. The first adapter 211 is an adapter to which an operation cable 301 is detachably connected. The second adapter 212 is an adapter to which the first attachment/detachment portion 1501 of the endoscope 100 is detachably connected.

The operation-receiving portion 220 receives an operation input from the operation device 300 via the operation cable 301. In a case where the operation device 300 and the driving device 200 communicate by wireless communication instead of wired communication, the operation-receiving portion 220 has a known wireless-receiving module.

The air supply suction drive portion 230 is connected to the air supply suction tube 172 inserted into the internal path 101 of the endoscope 100. The air supply suction drive portion 230 includes a pump and the like, and supplies air to the air supply suction tube 172. Further, the air supply suction drive portion 230 sucks air from the air supply suction tube 172.

The wire drive portion 250 has a drive portion and an encoder (not shown). The drive portion pulls or relaxes the curved wire 160 (upper curved wire 161u, lower curved wire 161d, left curved wire 161l, right curved wire 161r) by a pulley or the like. The encoder detects a traction amount of the curved wire 160. The detection result of the encoder is acquired by the drive controller 260 of the driving device 200.

The drive controller 260 controls the entire driving device 200. The drive controller 260 acquires the operation input received by the operation-receiving portion 220. The drive controller 260 controls the air supply suction drive portion 230 and the wire drive portion 250 based on the acquired operation input. Specifically, the drive controller 260 calculates a control amount (wire traction amount, wire tension, or the like) in which the wire drive portion 250 drives the curved portion 112 or the like, from the operation input to the curved portion 112 or the like received by the operation-receiving portion 220, based on a changeable control parameter and a conversion formula having the control parameter as a coefficient. The control parameter is not limited to the coefficient of the conversion formula, and may be a parameter that replaces a part or all of the conversion formula.

The drive controller 260 is a computer capable of executing a program including a processor, a memory, a storage capable of storing programs and data, and an input/output controller. The function of the drive controller 260 is realized by the processor executing the program. At least some functions of the drive controller 260 may be realized by a dedicated logic circuit.

Since the drive controller 260 controls a plurality of motors for driving a plurality of curved wires 160 with high accuracy, it is desirable that the drive controller 260 have high calculation performance.

The drive controller 260 may further have a configuration other than the processor, the memory, the storage, and the input/output controller. For example, the drive controller 260 may further have an image calculation portion that performs some or all of the image processing and image recognition processing. By further having the image calculation portion, the drive controller 260 can execute specific image processing and image recognition processing at high speed. The image calculation portion may be mounted on a separate hardware device connected by a communication line.

### [Operation Device 300]

The operation device 300 is a device for inputting an operation for driving the endoscope 100. The input operation input is transmitted to the driving device 200 via the operation cable 301.

### [Video Control Device 500]

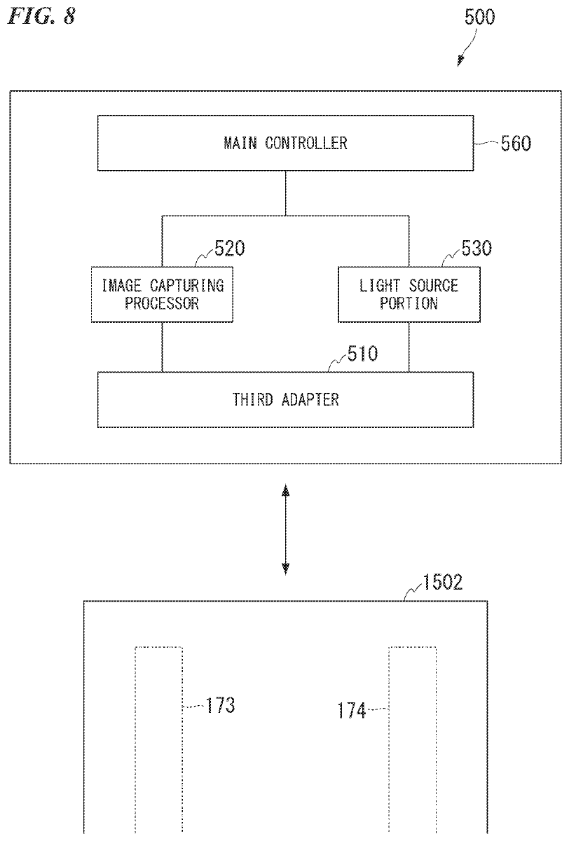

FIG. 8 is a functional block diagram of the video control device 500.

The video control device 500 controls the electric endoscope system 1000. The video control device 500 includes a third adapter 510, an imaging processor 520, a light source portion 530, and a main controller 560.

The third adapter 510 is an adapter to which the second attachment/detachment portion 1502 of the endoscope 100 is detachably connected.

The imaging processor 520 converts the imaging signal acquired from the imaging portion 111c of the distal end portion 111 into a captured image via the imaging cable 173.

The light source portion 530 generates illumination light to be applied to the imaging target. The illumination light generated by the light source portion 530 is guided to the illumination portion 111b of the distal end portion 111 via the light guide 174.

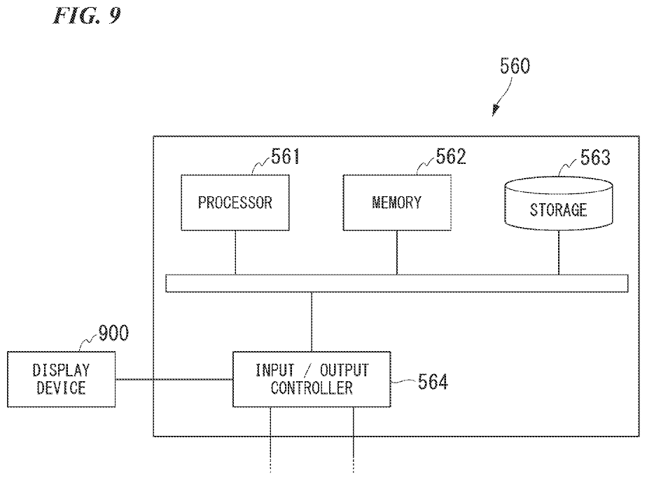

FIG. 9 is a functional block diagram of the main controller 560.

The main controller 560 is a computer capable of executing a program equipped with a processor 561 and a memory 562 or the like. The function of the main controller 560 is realized by the processor 561 executing the program. At least some functions of the main controller 560 may be realized by a dedicated logic circuit.

The main controller 560 has a processor 561, a memory 562 that can read the program, a storage 563, and an input/output controller 564.

The storage 563 is a non-volatile recording medium that stores the above-mentioned program and necessary data. The storage 563 is composed of, for example, a ROM, a hard disk, or the like. The program stored in the storage 563 is read into the memory 562 and executed by the processor 561.

The input/output controller 564 is connected to the imaging processor 520, the light source portion 530, the driving device 200, a measurement device 800, the display device 900, an input device (not shown), an external camera (not shown), and a network device (not shown). The input/output controller 564 transmits/receives data to/from the connected device and transmits/receives control signals based on the control of the processor 561.

The main controller 560 can perform image processing on the captured image acquired by the imaging processor 520. The main controller 560 can generate a GUI image or a CG image for the purpose of providing information to the operator S. The main controller 560 can display the captured image, GUI image, and CG image on the display device 900.

The main controller 560 is not limited to an integrated hardware device. For example, the main controller 560 may be configured by partially separating the hardware devices as separate hardware devices and then connecting the separated hardware devices via a communication line. For example, the main controller 560 may be a cloud system that connects the separated storage 563 with a communication line.

The main controller 560 may further have a configuration other than the processor 561, the memory 562, the storage 563, and the input/output controller 564 shown in FIG. 9. For example, the main controller 560 may further have the image calculation portion that performs some or all of the image processing and the image recognition processing performed by the processor 561. By further having the image calculation portion, the main controller 560 can execute specific image processing and image recognition processing at high speed. The image calculation portion may be mounted on a separate hardware device connected by a communication line.

### [Operation of Electric Endoscope System 1000]

Next, the operation of the electric endoscope system 1000 of this embodiment will be described. Specifically, the operation of the user calibrating the endoscope 100 before use in the operating room or the treatment room will be described.

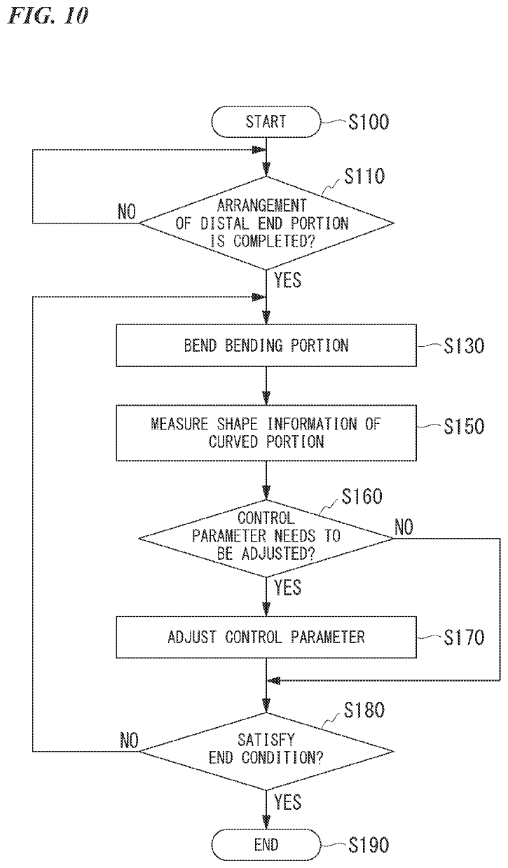

Hereinafter, a description will be given according to the control flowchart of the main controller 560 of the control device 600 shown in FIG.10. When the control device 600 is activated, the main controller 560 starts control after performing initialization (step S100). Next, the main controller 560 (mainly the processor 561) performs step S110.

### <Step S110: Arrangement Step>

In step S110, the main controller 560 displays the GUI image on the display device 900 instructing the user to hang the endoscope 100 on the hanger 710 and suspend a distal end portion 180 of the endoscope 100 including the curved portion 112 from the hanger 710.

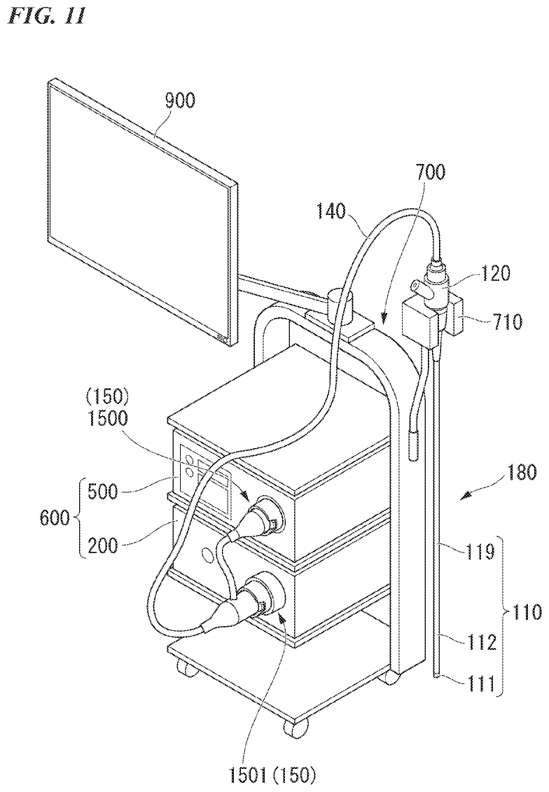

FIG. 11 is a diagram showing an endoscope 100 or the like in which the distal end portion 180 is suspended.

The user hangs the distal end portion 180 from the hanger 710 according to the instructions displayed on the GUI image. Specifically, the user suspends and arranges the distal end portion 180 so that the distal end portion 180 does not come into contact with the floor or the like and the distal end portion 180 is in a straight line state along the vertical direction. In the following description, the state of the suspended distal end portion 180 is also referred to as an “initial state” or a “natural hanging state”.

The distal end portion 180 is a portion of the distal end side Al of the endoscope 100 including at least the curved portion 112. The distal end portion 180 may include part or all of the internal soft portion 119. Further, a part or all of the extracorporeal soft portion 140 may be included.

In the example shown in FIG. 11, the connecting portion 120 is hung on the hanger 710, and the insertion portion 110 is hung as the distal end portion 180. The internal soft portion 119 may be hung on the hanger 710, and a part of the internal soft portion 119, the curved portion 112, and the distal end portion 111 may be suspended as the distal end portion 180. The extracorporeal soft portion 140 may be hung on the hanger 710, and a part of the extracorporeal soft portion 140, the internal soft portion 119, the curved portion 112, and the distal end portion 111 may be suspended as the distal end portion 180.

The main controller 560 detects whether the distal end portion 180 of the endoscope 100 is in the initial state. The main controller 560 may detect whether the distal end portion 180 is in the initial state by causing the user to input information indicating the completion of the arrangement step from an input device (not shown). Further, the main controller 560 may detect whether the distal end portion 180 is in the initial state based on the captured image captured by the imaging portion 111c of the endoscope 100, the sensor mounted on the endoscope 100, or the like. Further, the main controller 560 may detect whether the distal end portion 180 is in the initial state based on an image taken by an external camera (not shown) connected to the video control device 500. Next, the main controller 560 performs step S130.

### <Step S130: Bending Step>

In step S130, the main controller 560 controls the drive controller 260 so that the control amount (wire traction amount, wire tension, or the like) of the curved portion 112 is the adjustment value V, and bends the curved portion 112 in at least one direction of the UD direction and the LR direction. Next, the main controller 560 performs step S150.

In step S130, the main controller 560 may estimate the bending direction in which the curved portion 112 is frequently used and is greatly affected by aging deterioration from the usage history of the endoscope 100, so as to bend the curved portion 112 in the estimated bending direction. As a result, the main controller 560 can efficiently calibrate the endoscope 100.

### <Step S150: Measurement Step>

The main controller 560 measures the shape information of the curved portion 112 in step S150. Specifically, the main controller 560 measures the shape information of the curved portion 112 based on the captured image captured by the imaging portion 111c of the endoscope 100, the sensor mounted on the endoscope 100, and the like. Further, the main controller 560 may measure the shape information of the curved portion 112 based on an image taken by an external camera (not shown) connected to the video control device 500. Next, the main controller 560 performs step S160.

### <Step S160: Adjustment Step>

The main controller 560 compares the shape information of the curved portion 112 that is ideally curved when the control amount (wire traction amount, wire tension, or the like) is set to the adjustment value V in step S160, and the shape information measured in step S150. Here, the main controller 560 holds in advance the shape information of the curved portion 112 that is ideally curved when the control amount is set to a predetermined value.

When the compared shape information is different, the main controller 560 determines that the control parameter for driving the curved portion 112 by the drive controller 260 of the endoscope 100 needs to be updated, and then performs step S170. In a case where there is almost no difference in the compared shape information, the main controller 560 determines that it is not necessary to update the control parameters, and then performs step S180.

<Step S170: Adjustment Step>

The main controller 560 updates a control parameter used when calculating the control amount (wire traction amount, wire tension, or the like) required for the drive controller 260 of the endoscope 100 to form the curved portion 112 into a predetermined curved shape based on the shape information measured in the measurement step in step S170. The control parameters may be possessed by the drive controller 260 or the main controller 560.

The main controller 560 selects an optimum control parameter from a plurality of control parameters prepared in advance based on the above shape information. For example, the main controller 560 may select the optimum control parameter by using a learned model (machine learning model) in which the relationship between the shape information and the control parameter is learned in advance by machine learning. Further, the main controller 560 may select the optimum control parameter by referring to a database in which the relationship between the shape information and the control parameter is recorded in advance. Next, the main controller 560 performs step S180.

### <Step S180: End Determination Step>

In step S180, the main controller 560 determines the end of calibration of the endoscope 100. For example, in a case where a calibration step (bending step, measurement step, and adjustment step) is performed in each of the UD direction and the LR direction, the main controller 560 determines that the calibration of the endoscope 100 has been completed, and performs step S190 to end the control. In a case where it is determined that the calibration of the endoscope 100 has not been completed, the main controller 560 performs step S130 again.

According to the electric endoscope system 1000 of the present embodiment, the endoscope 100 can be easily calibrated without using a special calibration jig for fixing the curved portion 112, in a state where the endoscope 100 is normally stored, that is, in a state where the endoscope 100 is hung on the hanger 710. Therefore, even when the operating room or treatment room in which the electric endoscope system 1000 is installed is small, the control device 600 (mainly the main controller 560) can easily calibrate the endoscope 100.

The distal end portion 180 of the endoscope 100 is suspended so as to be in a straight line along the vertical direction in the initial state. Therefore, the shape of the distal end portion 180 of the endoscope 100 in the initial state is uniquely determined without using a special calibration jig. Therefore, since the control device 600 (mainly the main controller 560) can perform the calibration step based on the uniquely determined initial shape, the endoscope 100 can be accurately calibrated under the same conditions regardless of the environment in which it is used or the user who uses it.

Although the first embodiment of the present disclosure has been described in detail with reference to the drawings, the specific configuration is not limited to this embodiment, and includes design changes and the like within a range that does not deviate from the gist of the present disclosure. In addition, the components shown in the above-described embodiments and modifications can be appropriately combined and configured.

### Second Embodiment

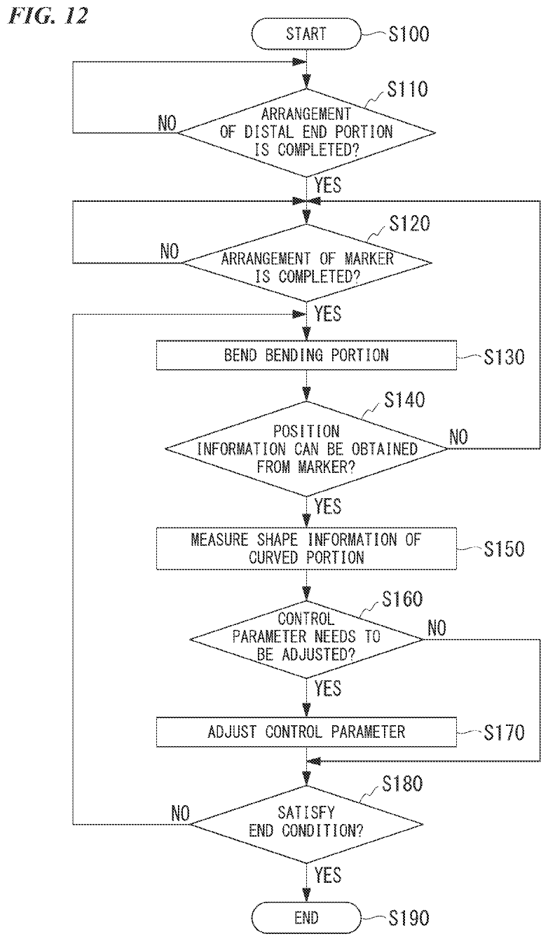

An electric endoscope system 1000B according to a second embodiment of the present disclosure will be described with reference to FIG. 12 to FIG. 14. In the following description, the same reference numerals will be given to the configurations common to those already described, and duplicate description will be omitted.

### [Electric Endoscope System 1000B]

As shown in FIG. 1, the electric endoscope system 1000B has the same configuration as the electric endoscope system 1000 of the first embodiment. The electric endoscope system 1000B differs from the electric endoscope system 1000 of the first embodiment only in the operation of using a marker.

Hereinafter, the description will be given according to the control flowchart of the main controller 560 of the control device 600 shown in FIG.12. When the control device 600 is activated, the main controller 560 starts control after performing initialization (step S100). Next, the main controller 560 (mainly the processor 561) performs step S110.

### <Step S110: Arrangement Step>

In step S110, the main controller 560 implements the arrangement step in the same manner as in the first embodiment. Next, the main controller 560 performs step S120.

<Step S120: Marker Arrangement Step>

In step S120, the main controller 560 displays the GUI image instructing the user to place the marker M on the display device 900. The marker M has a known marker pattern m capable of specifying relative position information. The marker pattern m is a pattern in which relative position information can be specified by observing from different places.

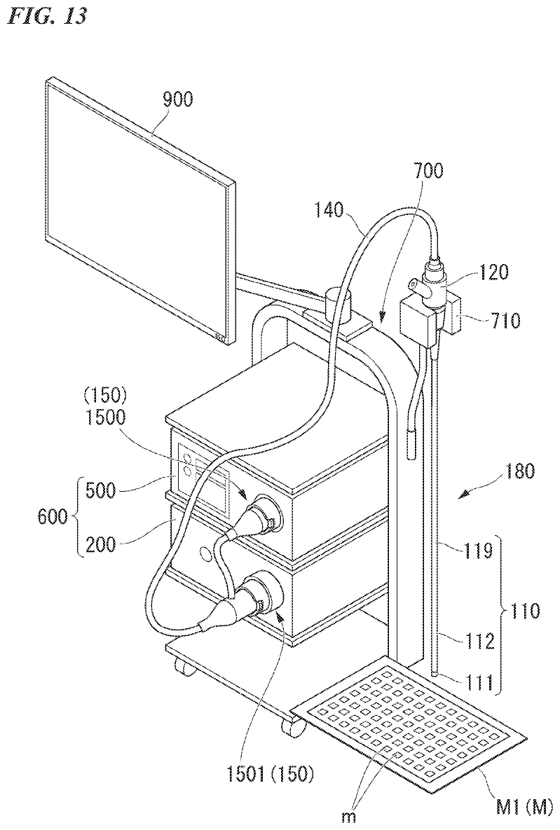

FIG. 13 is a diagram showing a sheet-shaped marker M1 as an example of the marker M.

The marker M1 is a sheet-shaped marker having a known marker pattern m. The user installs the marker M1 below the suspended distal end portion 180 in the vertical direction so as to form a straight line along the vertical direction. Since the field of view of the imaging portion 111c of the endoscope 100 is downward in the vertical direction, the imaging portion 111c can image the marker M1 and acquire relative position information from the marker M.

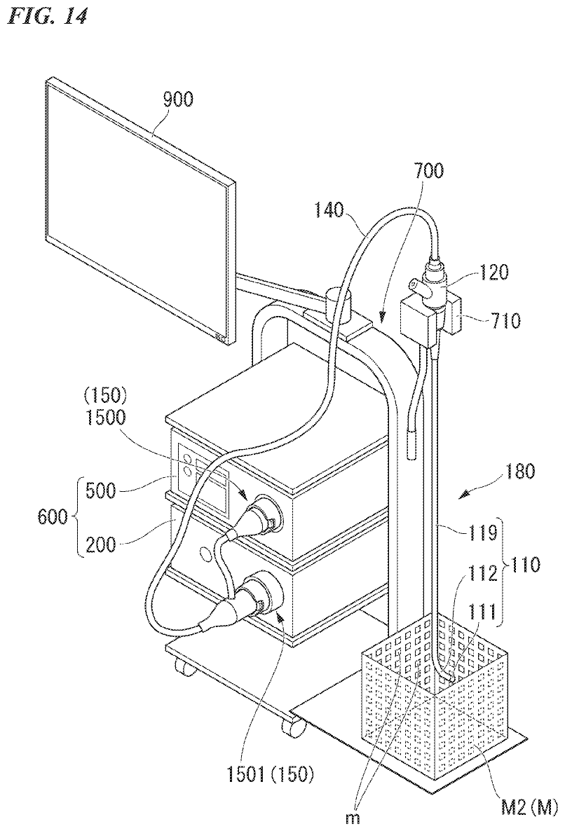

FIG. 14 is a diagram showing a box-shaped marker M2 as an example of the marker M.

The marker M installed by the user may be a box-shaped marker M2 having a plurality of surfaces having the marker pattern m. The marker M2 is formed in a box shape that opens upward in the vertical direction, and has a known marker pattern m on the inner surface. The user installs the marker M2 vertically below the suspended distal end portion 180 so that the distal end portion 111 of the endoscope 100 is arranged inside the box-shaped marker M2. Since the field of view of the imaging portion 111c of the endoscope 100 is downward in the vertical direction, the imaging portion 111c can image the marker M2 and acquire relative position information from the marker M.

The main controller 560 detects that the marker M has been arranged. The main controller 560 may detect that the marker M has been arranged by causing the user to input information indicating the completion of the marker arrangement step from an input device (not shown). Further, the main controller 560 may detect that the marker M is arranged based on the captured image or the like captured by the imaging portion 111c of the endoscope 100. Next, the main controller 560 performs step S130.

### <Step S130: Bending Step>

The main controller 560 performs the bending step in step S130 as in the first embodiment. Next, the main controller 560 performs step S140.

### <Step S140: Marker Position Information Acquisition Step>

In step S140, the main controller 560 images the marker M by the imaging portion 111c, and acquires the relative position information from the marker M based on the captured image. In a case where the imaging portion 111c cannot image the marker M or cannot acquire the relative position information from the marker M, there is a possibility that the position of the marker M is inadequate, so the main controller 560 displays the GUI image instructing the user to reposition the marker M on the display device 900, and performs step S120 again. The user repositions the marker M so that the marker pattern m is included in the field of view of the imaging portion 111c both before and after the curved portion 112 is curved. For example, the main controller 560 may estimate the direction in which the curved portion 112 is frequently used and is greatly affected by aging deterioration from the usage history of the endoscope 100, and display the GUI image on the display device 900 instructing the user to reposition the marker M so that the marker pattern m is included in the field of view of the imaging unit 111c before and after bending in the estimated bending direction.

In a case where the curved portion 112 is largely curved in the bending step, the marker M is preferably a box-shaped marker M2 rather than a sheet-shaped marker Ml. This is because in the box-shaped marker M2, there is a high possibility that the marker pattern m is included in the field of view of the imaging portion 111c both before and after the curved portion 112 is curved.

If the main controller 560 can acquire the relative position information from the marker M in step S140, then the main controller 560 performs step S150.

### <Step S150: Measurement Step>

The main controller 560 measures the shape information of the curved portion 112 in step S150. Specifically, the shape information of the curved portion 112 is measured by using the relative position information from the acquired marker M. Next, the main controller 560 performs step S160 and subsequent steps as in the first embodiment.

According to the electric endoscope system 1000B of this embodiment, since the control device 600 (mainly the main controller 560) can perform the calibration step based on the uniquely determined initial shape, the endoscope 100 is accurately calibrated under the same conditions regardless of the environment in which it is used or the user who uses it. Further, the control device 600 (mainly the main controller 560) can measure the shape information of the curved portion 112 more accurately by using the marker M, and can calibrate the endoscope 100 more accurately.

The second embodiment of the present disclosure has been described in detail with reference to the drawings, but the specific configuration is not limited to this embodiment, and includes design changes and the like within a range that does not deviate from the gist of the present disclosure. In addition, the components shown in the above-described embodiments and modifications can be appropriately combined and configured.

### MODIFICATION EXAMPLE 1

In the above embodiment, the endoscope 100 of the electric endoscope system 1000 and the operation device 300 are separated. However, the modes of the endoscope 100 and the operation device 300 are not limited to this. The method for calibrating an endoscope according to the present disclosure may be applied to a conventional endoscope system in which an endoscope and an operation portion are integrated. In that case, for example, the operating portion of the conventional endoscope is hung on the hanger 710, and the insertion portion on the distal end side is hung as the distal end portion.

Each embodiment may be realized by recording the program on a computer-readable recording medium, loading the program recorded on the recording medium into a computer system, and executing the program. The “computer system” includes hardware such as an OS and peripheral devices. Further, the “computer-readable recording medium” refers to a portable medium such as a flexible disk, a magneto-optical disk, a ROM, or a CD-ROM, or a storage device such as a hard disk built in a computer system. Further, a “computer-readable recording medium” may include a medium that dynamically holds a program for a short period of time, such as a communication line when a program is transmitted via a network such as the Internet or a communication line such as a telephone line, and a medium that holds a program for a certain period of time, such as volatile memory inside a computer system that serves as a server or client in that case. Further, the above-mentioned program may be a program for realizing some of the above-mentioned functions, and may be a program for realizing the above-mentioned functions in combination with a program already recorded in the computer system.

What is claimed is:

1. A method for calibrating an endoscope having a curved portion capable of being driven by bending, the method comprising:
an arrangement step of suspending and arranging a distal end portion of the endoscope including the curved portion;
a bending step of bending the curved portion of the distal end portion which has been suspended;
a measurement step of measuring shape information of the curved portion; and
an adjustment step of updating a control parameter by which the endoscope drives the curved portion, based on the shape information which has been measured.

2. The method for calibrating the endoscope according to claim 1, wherein, in the arrangement step, the distal end portion is suspended and arranged so that the distal end portion is in a straight line along a vertical direction.

3. The method for calibrating the endoscope according to claim 1, wherein the control parameter, which has been updated in the adjustment step, is a parameter used when calculating a control amount for driving the curved portion from an operation input for the curved portion.

4. The method for calibrating the endoscope according to claim 1, wherein, in the measurement step, the shape information of the curved portion is measured based on a sensor included in the endoscope.

5. The method for calibrating the endoscope according to claim 1, further comprising:
a marker arrangement step of arranging a marker having a marker pattern capable of specifying relative position information downward in a vertical direction with respect to the distal end portion, and
in the measurement step, the shape information of the curved portion is measured based on an image obtained by capturing the marker pattern of the marker.

6. The method for calibrating the endoscope according to claim 5, wherein the marker is formed in a sheet shape.

7. The method for calibrating the endoscope according to claim 5, wherein the marker includes a plurality of surfaces having the marker pattern.

8. An endoscope system comprising:
an endoscope having a curved portion capable of being driven by bending; and
a control device configured to control driving of the curved portion,
wherein the control device performs
a bending step of bending the curved portion,
a measurement step of measuring shape information of the curved portion, and
an adjustment step of updating a control parameter by which the endoscope drives the curved portion, based on the shape information which has been measured.

9. The endoscope system according to claim 8, wherein the control device performs, before the bending step, an arrangement step of confirming that the distal end portion of the endoscope including the curved portion is suspended and arranged.

10. The endoscope system according to claim 9, wherein the control device performs, before the bending step, a marker arrangement step of confirming that a marker having a marker pattern capable of specifying relative position information is arranged below the distal end portion in a vertical direction.

11. The endoscope system according to claim 10, wherein, in a case where the endoscope cannot image the marker pattern both before and after bending the curved portion in the bending step, the control device instructs a user to rearrange the marker.
